(19) United States
(12) Patent Application Publication
(10) Pub. No.: US 2010/0298916 A1
(43) Pub. Date: Nov. 25, 2010

(54) Pelvic implanted neural electrode and method for implanting same

(76) Inventors: Pierre RABISCHONG, Montpellier (FR); Stephane Gerard GALLEGO, Lyon (FR); Georges FRAISSE, Antibs (FR)

Correspondence Address: Thomas R. Vigil Law Offices, 319 Bluff Court, Barrington, IL, 60010, US

(21) Appl. No.: 12/454,820

(22) Filed: May 22, 2009

(51) Int. Cl. A61N 1/05

(52) U.S. Cl. 607/116

(57) ABSTRACT

The implantable monopolar electrode assembly includes a base support structure including a layer made of a nickel titanium alloy having a temperature memory. The method is directed to electro-stimulation of muscles and to a method of implanting a monopolar electrode assembly. The method includes the steps of: first performing an endoscopic approach to the nerves and then selective electro stimulation by performing a neurolysis on nerve trunks until a desired target nerve fascicle and associated muscle is located, followed by insertion of the monopolar electrode assembly adjacent the target nerve fascicle for subsequent connection to a neurposthesis system.

### BACKGROUND OF THE INVENTION

1. Field of the Invention

The present invention relates to electro-stimulation of muscles and more specifically to a monopolar electrode for such stimulation and a method of implanting such an electrode by first performing an endoscopic approach of the nerves and then selective electro stimulation by performing a neurolysis on nerve trunks.

2. Description of the Prior Art

Electro stimulation has been used for many years to activate muscles artificially by controlling three parameters: 1) frequency in Herz, 2) intensity in milliampere and 3) pulse width in microsecond. The clinical applications were made principally on paralysed patients from cerebral origin like in hemiplegia or from spinal cord origin like in tetraplegia (paralysis of the four limbs by cervical spine lesion) or paraplegia (lower limbs paralysis by thoracic level lesion). The electrical stimulation can be applied by electrodes placed on the skin (cutaneous), on the surface of the muscles at the motor point (epimysial), within the muscles (intramuscular), on the nerves (neural), within the fascicules of a nerve (intrafascicular), on the spinal roots (radicular) or on the spinal cord (epidural).

The technical devices used in the case of implantation are always the same: electrodes, electronic implant, antenna transferring power and signal and external programmer or controller doing what the brain is normally doing: choosing the right muscle at the right time.

Many researchers in the world have been trying to restore motor functions by FES (functional electrical stimulation), first with surface electrodes and then with an implant that can be called a neuroprosthesis. The domain is very large and it is possible to identify: the motor neuroprosthesis in order to restore walking and grasping, the sensorial neuroprosthesis for blind, deaf and pain, the visceral neuroprosthesis for heart (pacemakers), bladder or colon.

A few teams are very active in this field, such as: Professors Mortimer and Peckham from Cleveland who created the Neurocontrol Company producing the Free Hand System to restore grasping in tetraplegic patients and the Free Stand System to restore locomotion. But this company closed down a few years ago and the production of this system has stopped.

Many companies that produce cochlear implants were using the same technology to produce motor neuroprosthesis. That is the case for Nucleus in Australia which has the largest part of the cochlear implant market and MXM from Antibes in France.

At the moment, the Alfred Mann Foundation in California is producing a very interesting component called BION which was designed by G. Loeb from Toronto, Canada and Joseph Schulman from the US. Their present version is made with a 16 mm length electronic component which is a single channel microstimulator acting in monopolar mode and activated by a radiofrequency link. Another version is equipped with a battery and is called FES BION.

In France, at the Faculty of Medicine in Montpellier and jointly with a Biomechanical Research Unit belonging to INSERM (French National Institute of Health and Medical Research) an implant was designed having epimysial electrodes and was used with two paraplegic patients.

A method for restoring walking in paraplegic patients was initiated in 1996 and applied to two patients in 1999. It involved using implanted electro stimulation made by three components: first, an electronic implant able to distribute electric current to the different targets, nerves and muscles, at the right time, second, specific electrodes, are placed at the surface of the muscles as close as possible to the motor point (epimysial electrodes) or placed around a nerve or a fascicle of a nerve (neural electrode) with low tension from 0.8 v to 12 v using a cuff electrode having a diameter from 0.5 mm to 10 mm and third, an outside equipment made by an antenna placed on the skin in the front of the internal antenna of the implant transferring the electrical power and the signal and connected with an external programmer which is doing what the brain is normally doing: activating the right muscle at the right time. The implantation of electrodes requires an open surgery with different incisions corresponding to the muscular targets, representing a potential risk of infection as well as skin scars which can be aesthetically critical. It was observed with the two operated patients that the epimysial electrodes are relatively difficult to put in the optimal place in relation with the motor point which can be multiple in big muscles like the gluteus maximus. It was also noted that having too many wires travelling within the body is a risk to carry an infection, as observed in one of the patients who got an infection due to a small wire emerging outside of the skin a long time after the surgical procedure and collecting a germ from the urinary pubic region forcing the surgical team to remove all the implanted devices even if an antibiotic treatment was employed.

Listed below are several prior art patents disclosing the use of cuff electrodes for providing electro-stimulation to nerves.

| U.S. Pat. No. | Patentee |
|---|---|
| 5,038,781 | Lynch |
| 5,167,229 | Peckham et al. |
| 6,163,725 | Peckham et al. |
| 6,718,210 | Peckham et al. |

The main technical problem in neuromuscular stimulation is to be able to put in place the electrodes with a minimal invasive procedure.

### SUMMARY OF THE INVENTION

According to the teachings of the present invention there is provided a method for the electro-stimulation of muscles and a method for implanting a monopolar electrode. The method includes the steps of: first performing an endoscopic approach to the nerves and then selective electro stimulation by performing a neurolysis on nerve trunks until a desired target nerve fascicle and associated muscle is located, followed by insertion of a monopolar electrode adjacent the target nerve fascicle for subsequent connection with a connection wire having a steel spring and a platinum wire to a neurposthesis system. Preferably, the endoscopic approach is in the stomach and the stomach is first inflated with air. The monopolar electrode for electrostimulation of a fascicle in a nerve bundle comprises a bared end of an insulated wire which is fixed to an insulated base structure adapted to be positioned adjacent a fascicle in a nerve bundle. In one embodiment, the base support is made of insulated Nitinol which is soft below the temperature of a human body and which forms a curled spiral shape at the temperature of a human body.

### BRIEF DESCRIPTION OF THE SEVERAL VIEWS OF THE DRAWINGS

FIG. 1 is perspective view with a portion broken away of a peripheral pelvic nerve and shows a plurality of fascicles of the nerve.

FIG. 2 is a sectional view of one fascicle of nerves.

FIG. 3 illustrates a surgical neurolysis of a nervous trunk.

FIG. 4 illustrates a procedure for identifying a nerve in a fascicle of nerves by bipolar stimulation.

FIG. 5 is a perspective view of a monopolar neural electrode constructed according to the teachings of the present invention.

FIG. 6 is a perspective view of the monopolar neural electrode mounted to a nervous trunk and in electrical contact with a selected nerve.

FIG. 7 is a plan view of a memory shape electrode assembly constructed according to the teachings of the present invention.

FIG. 8 is a plan view of the memory shape electrode after it has been placed around a nerve and warmed to body temperature to cause the electrode to assume a curled spiral shape about the nerve.

FIG. 9 is a cross-sectional view of the electrode shown in FIG. 7 and is taken along line 9-9 in FIG. 7.

FIG. 10 is a perspective view of another embodiment of an electrode, constructed according to the teachings of the present invention, around one fascicle and fixed by sutures to adjacent tissue.

FIG. 11 is a perspective view of a nerve trunk with a portion broken away to show fascicle and showing still another embodiment of two electrodes, constructed according to the teachings of the present invention, positioned around two respective fascicle.

FIG. 12 is a perspective view of one of the three layer electrodes shown in FIG. 11.

FIG. 13 is a cross-sectional view of the electrode shown in FIG. 12 with a fascicle show within the electrode and is taken along line 13-13 in FIG. 12.

FIG. 14 is a cross-sectional view of the electrode shown in FIG. 13 closed around the fascicle, but, for illustrative purposes, with the fascicle shown adjacent the electrode.

FIG. 15 is a perspective back view of the electrode shown in FIG. 13, is taken along line 15-15 of FIG. 13 and shows an opening in the back of the electrode for a wire conductor to extend into the three layer electrode.

FIG. 16 is a perspective view of an open cuff electrode assembly.

FIG. 17 is a perspective view of a semi-open cuff electrode assembly.

FIG. 18 is perspective plan view of a platinum strip for use in a lasso electrode assembly.

FIG. 19 is a perspective view of the lasso electrode assembly folded over for capturing a nerve.

FIG. 20 is a perspective view of the lasso electrode assembly around a nerve.

### DESCRIPTION OF THE PREFERRED EMBODIMENTS

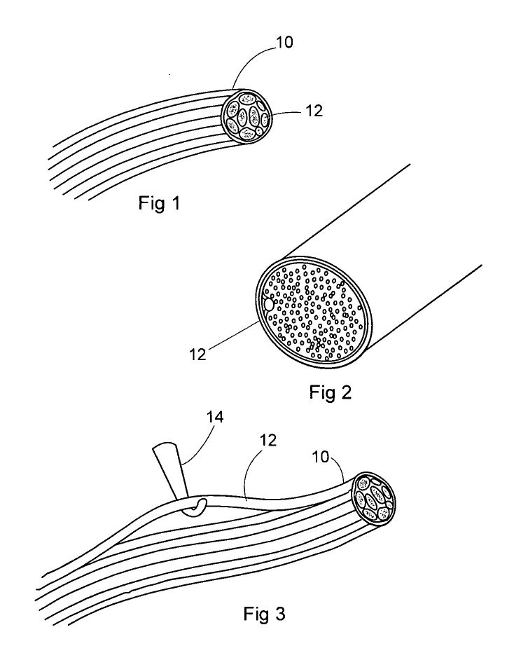

Referring now to the drawings in greater detail, a peripheral pelvic nerve bindle 10 is shown in FIG. 1 cut away to show a plurality of fascicles 12 of the nerve 10. A cross section of one fascicle 12 is shown in FIG. 2.

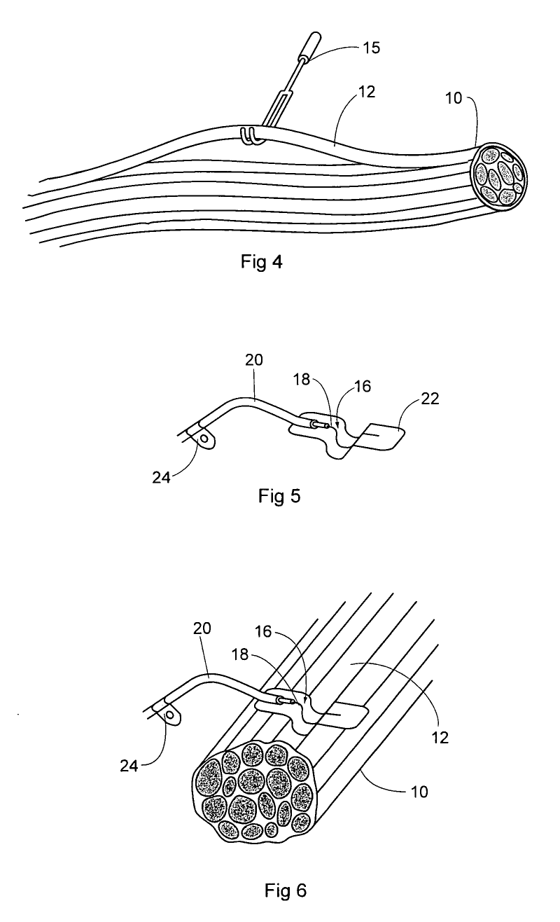

According to the teachings of the present invention individual fascicles 12 are selected from the nerve bundle 10 with a tool 14 as shown in FIG. 3 or a tool 15 as shown in FIG. 4 and supplied with a stimulus to determine which muscle it controls, i.e., the target. This process is repeated until a desired fascicle 12 is located.

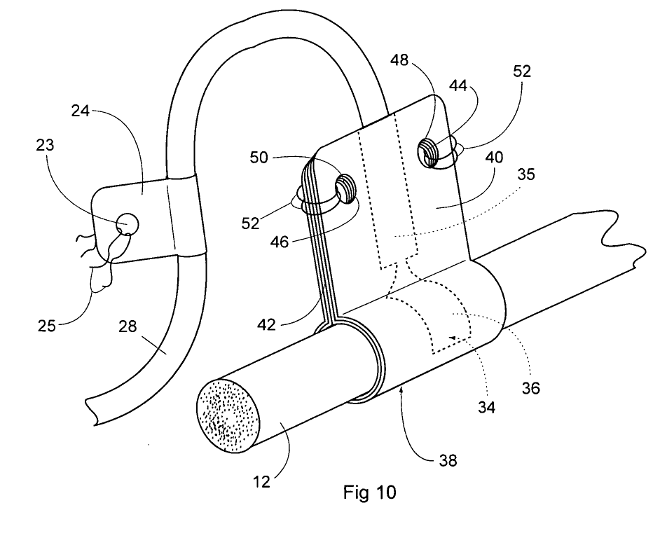

Once the desired fascicle 12 is found, a monopolar electrode 16 including a bare wire conductor end 18 of an insulated wire conductor 20 fixed to a U-shaped and winged insulating base support 22 (like a BX or conduit strap in shape), as shown in FIG. 5, is positioned around the desired fascicle 12 as shown in FIG. 6. The base support 22 has the general shape of a conduit strap or clamp. A fixation clamp 24 is mounted on the wire 20 for use in securing the electrode 16 to surrounding tissue. This clamp is secured to adjacent tissue by sutures 25 that extend through an opening 23 in the clamp 24, as shown in FIG. 10.

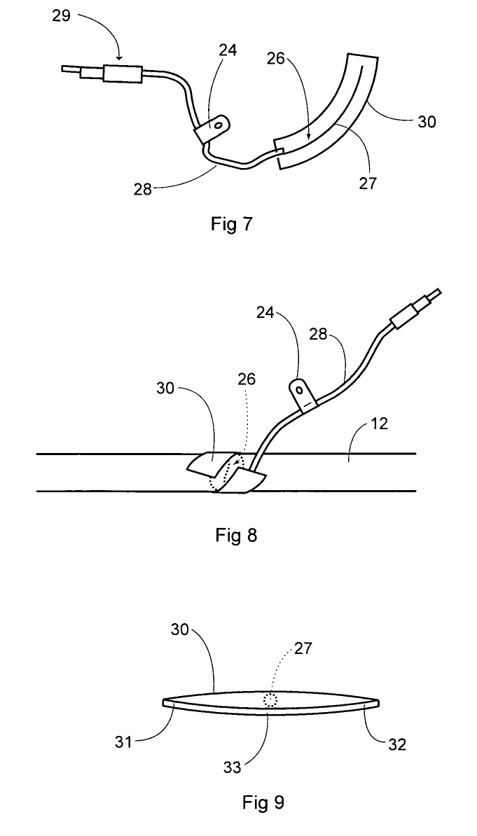

An alternative form of monopolar electrode 26 is shown in FIG. 7. the electrode 26 comprises a bare wire end 27 at the inner end of an insulated wire conductor 28. The wire conductor 28 has a male prong connector 29 at it's outer end. The bare wire end 27 is mounted on a curved strip 30 of memory material forming a base support structure 30 and being insulated on the back side as shown in FIG. 7. The memory material is preferably a nickel titanium alloy known as Nitinol. Nitinol has a temperature shape memory where, at a lower temperature, the strip 30 is soft with a flat shape and easily formed and where, at a higher temperature which is at or below the normal temperature of a human body, the strip 30 assumes a harder, curled shape, embracing a fascicle 12 adjacent which the strip 30 is positioned, as shown in FIG. 8.

FIG. 9 shows a cross section of the strip 30 with two wings 31 and 32 and a coating of insulation 33 on the backside of the strip 30.

FIG. 10 shows another embodiment of a metal electrode 34 including a straight electrode portion 35 and an arcuate electrode portion 36 which is held around a fascicle 12 by a double layer, pipe hanger shaped, clamp 38. The clamp 38 includes an outer layer of flexible insulating material 40 which is adhered to an inner metal layer 42 which defines a base support, which can be made of Nitinol, which initially can have an open shape for being inserted around the fascicle 12 or which can be made of insulating material. The arcuate electrode portion 36 can be fixed to the layer 42, can be made of Nitinol or can be flexible for being conformed by a Nitinol layer 42 around and in contact with the fascicle 12. The clamp has a generally pipe hanger shape with a generally cylindrical portion which is received around a fascicle 12 and two, parallel, leg portions which extend radially outwardly from the cylindrical portion for attaching the clamp 38 to adjacent tissue.

The flat electrode portion 35 is connected to the insulated wire conductor 28 which has a clamp 24 thereon for being sutured to adjacent tissue by sutures 25 extending through opening 23 in the clamp 24.

The layer 40 and the layer 42 of the clamp 38, in the portions thereof extending from the fascicle 12, have mating openings 44, 48 and 46, 50 on either side of the straight electrode portion 35 for receiving sutures 52 for securing the clamp 38 and electrode 36 to adjacent tissue.

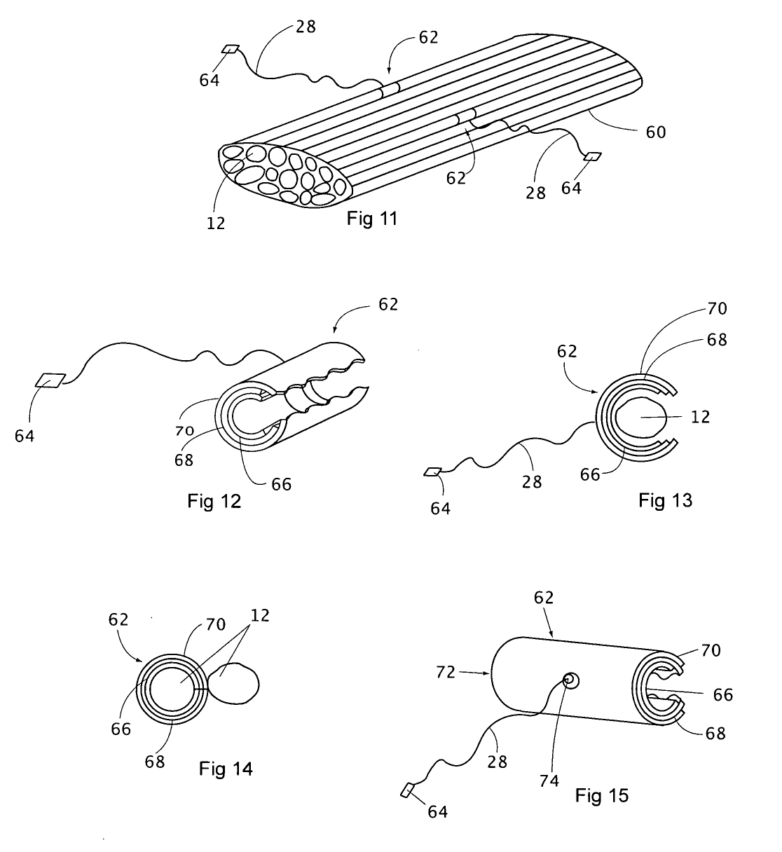

A perspective view of a nerve trunk 60 of fascicle 12 is shown in FIG. 11 and two electrode assemblies 62, constructed according to the teachings of the present invention are shown positioned, respectively, around two of the fascicle 12.

Each electrode assembly 62 is connected by an insulated wire conductor 28 to a connector 64 for connection to a source of electrical stimulation voltage as shown in FIGS. 11-13 and 15.

As shown in FIGS. 13-15, each electrode assembly 62 is C-shaped and includes three sem-cylindical layers comprises an inner electrode layer 66 having a large surface area in contact with the fascicle 12, a silicon insulating layer 68 and an outer layer 70. The inner electrode layer 68 can be made of platinum or Nitinol or of another flexible metal. The outer layer can be made of Nitinol which, at a temperature lower than body temperature, is partially open, as shown in FIGS. 12, 13 and 15, and, when received around a fascicle 12, assumes the shape shown in FIG. 12. The layers 68 and 70 form a base support structure for the inner layer electrode 66.

FIG. 15 shows a back side 72 of the electrode assembly 62 with an opening 74 for receiving the bare end of the wire conductor into the electrode assembly 62 for connection to the inner electrode layer 66.

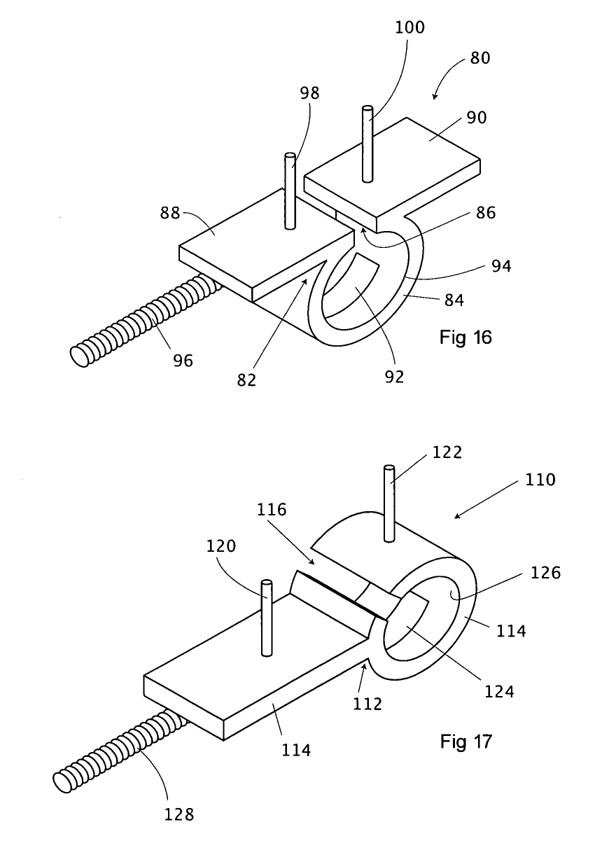

In FIG. 16 there is illustrated an open cuff monopolar electrode assembly 80 in the shape of a pipe clamp or BX cable clamp. The assembly 80 includes a insulating base support portion 82 made of an insulating material, e.g., plastic, and comprises a partially cylindrical portion 84 with a longitudinal slot 86 therein and opposed wing portions 88 and 90 which extend laterally outwardly from the slot 86 and from the cylindrical portion 84. A flat curved platinum electrode 92 is mounted on an inner cylindrical surface 94 of the partially cylindrical portion 84 positioned to make electrical contact with a nerve inserted into the partially cylindrical portion 84. A wire conductor 96 extends through the partially cylindrical portion near one wing portion 88 and is electrically connected to the flat platinum electrode 92. A removable rod or skirt 98, 100 is fixed to each wing 88 and 90 for being gripped to pull the wings away from the slot 86 to expand the slot 86 to enable a nerve to be inserted through the expanded slot 86 into the partially cylindrical portion 84, When the rods or skirts 98,100 are released the cylindrical portion gripps the nerve inserted therein and presses the platinum electrode against the nerve.

In FIG. 17 there is illustrated a semi-cuff monopolar electrode assembly 110 which includes an insulating base support portion 112 that is made of an insulating material, e.g., plastic and includes a partially cylindrical portion 114 with a longitudinal slot 116 therein. One wing 118 extends from a side of the partially cylindrical portion near the slot 116. A rod or skirt 120 is removably mounted to the wing 114 and a second rod or skirt 122 is removably mounted to the cylindrical portion near the slot 116 on a side of the partially cylindrical portion 114 which is on the other side of the slot 116 from where the wing 114 is fixed to the partially cylindrical portion 114. A flat curved platinum electrode 124 is mounted on an inner surface 126 of the partially cylindrical portion and a wire conductor 128 extends through the partially cylindrical portion adjacent the wing 118 for electrical connection to the flat platinum electrode. It will be understood that the rods or skirts 120,122 are gripped and pulled to open or expand the slot 116 to enable a nerve to be inserted through the expanded slot 116 into the partially cylindrical portion 114. Then the partially cylindrical portion is allowed to grip the nerve and urge the curved flat platinum electrode against the nerve.

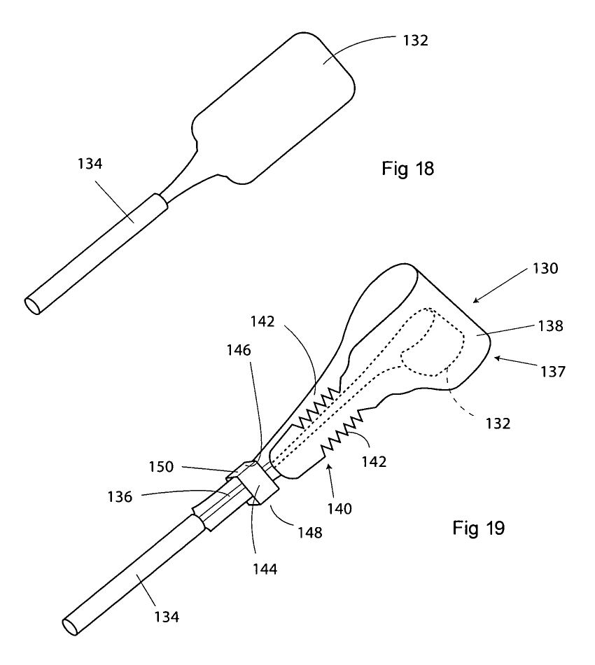

In FIGS. 18, 19 and 20 there is illustrated a lasso monopolar electrode assembly 130. In FIG. 18 a flat but bendable platinum electrode 132 electrically connected to a wire conductor 134. an approximately 0.05 mm wire 136 extends from the approximately 50 micron thick flat platinum electrode 132 into the coiled wire conductor 134.

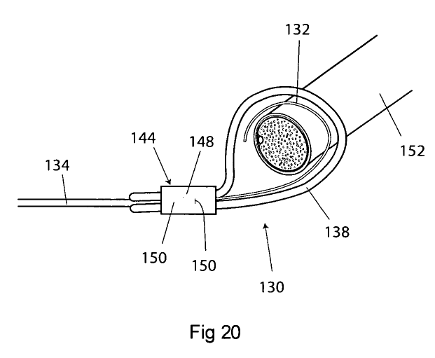

As shown in FIG. 19 the flat platinum electrode 132 is curled inside an insulating base support portion 137 comprising an insulating silicon flap 138. The silicon flap 138 has a narrowed end portion 140 which has serrations 142 on opposite side edges thereof and is adapted to be pulled through a silicon collar 144 which extends around a narrowed portion 146 of the silicon flap 138 and the platinum wire 136. Preferably the sides 146, 148 of the collar 144 has slits 150 therein for being engaged by the serrations 142 to lock the narrowed end portion 140 in the collar 144. As shown in FIG. 20, the narrowed end portion 140 is pulled through the collar 144 to lasso a nerve 152 that has been inserted in a loop created by the flat electrode 132 and the silicon flap 138 and press the flat curled electode 132 against the nerve 152.

The testing of fascicles 12 is preferably with an endoscopic approach which allows a very small incision and a very precise work by being close to the nerve with an excellent vision. In this procedure we go into the retroperitoneal space to find the appropriate nerves for the lower limb muscles as well as for the visceral organs placed in the pelvis: bladder, colon and rectum as well as the penis origin. With a team of gynaecologic surgeons, we can approach all those nerves within their pelvic journey. The monopolar electrode 16, 26, 36, 66, 80, 110 or 130 is used in order to be sure that the electrode 16, 26, 36, 66, 80, 110 or 130 will be applied directly and closely to the surface of the nerve without using a bipolar cuff which has many disadvantages particularly in relation with its size: too small with the risk of compression of the nerve or too large with the risk of loosing the contact with the nerve.

A hyper selective nerve stimulation is performed by doing a neurolysis at the level of the trunk of the nerve in order to isolate the right fascicle 12 corresponding to the expected target. In fact in the peripheral nerves, three types of nervous fibres can be found: the motor fibres alpha and gamma, the sensitive fibres and the vegetative fibres (sympathetic and parasympathetic). The organization of the distribution of the fibres, around 110000 for the upper limb, is made with the progressive inclusion of those fibres within a fascicle 12 (or bundle) surrounded by a very strong fibrous sheath called the perineurium. All those fascicles 12 are surrounded by a more lax fibrous sheath called epineurium. The closer one is to the distal part of a nerve, the more precise is the destination of the fascicle 12. Therefore it is easy and not risky in the hands of a good surgeon to separate by opening the epineurium the fascicles 12 and to identify by stimulation during surgery the right one corresponding to the right target. If no muscular activation occurs, that means that the fascicle 12 is a sensitive one. In addition as demonstrated by an anatomical research, it is always within the fascicle 12 a small artery feeding the nervous fibres and avoiding anoxia during the procedure of isolation of a fascicle 12. The fibrosis normally occurring after any surgical action was not excessive and not risky for the nerve conduction. The great advantage of this technique is for example in the case of the lower limb to be able to put an electrode for the quadriceps muscle within the trunk of the femoral nerve in the pelvis without opening the skin of the thigh or for the tibialis anterior muscle which is located in the leg to put the electrode on the trunk of the sciatic nerve in the pelvic gluteal area after neurolysis. All the precise surgical protocol for all the important lower limb muscles to be stimulated in order to restore standing and walking as well as the sympathetic and parasympathetic pelvic nerves for bladder, rectum and erection control exits and can be controlled with the implanted electrodes 16, 26, 36, 66, 80, 110 or 130.

The method of the present invention differs from the one used on the two patient in 1999 by the use of an endoscopic approach which is an invasive technique for the implantation of the electrodes. In fact, all the nerves for the innervation of the visceral organs located in the pelvis: bladder, colon and rectum and genital organs as well as all the nerves devoted to the lower limb like gluteal, femoral and ischiatic nerves are reachable by an endoscopic approach. This method comprises first inflating the abdominal cavity with air and second inserting through the abdominal wall by an opening of one to two centimetres an endoscope and the working channels to operate in perfect technical conditions. Therefore it is not possible by an open laparotomy to have the same quality of direct vision of the deep structures than with the endoscope equipped with optic zoom and different vision angles. In addition, the positive pressure within the cavity due to the air inflation is a positive factor reducing substantially the bleeding.

The first electrode implantation is devoted to the main nervous trunk like femoral nerve or ischiatic nerve in which it is possible without any risk if done properly to isolate by dissecting gently the epineurium surrounding the nerve the right neural fascicle 12 corresponding to the right muscular target to activate in the programme. Using an electro stimulation of the fascicle 12 in a bipolar mode during surgery can easily allow one to identify the nature of the fascicle 12 or muscular made by motor and proprioceptive fibres or sensitive. It is also needed to check carefully the effect of the fascicular stimulation in order to be sure that it corresponds to the muscle that is provided to activate. When the fascicle 12 is identified, the original procedure comprises using an open cuff electrode which electrically isolates the fascicle 12 among the other fascicles 12 of the nerve and has the great advantage not to do any compression of the nerve able to produce by anoxia a conduction trouble.

The use of a monopolar electrode 16, 26, 36, 66, 80, 110 or 130 is more simple to put in place and has the great advantage to assure a perfect contact with the nerve which is not the case for the bipolar cuff electrode for which it is needed to find the right shape otherwise if it is too small, it generates a compression of the fascicle 12 or, if it is too large, the contact of the two plots on the nerve can be wrong observed during experimental investigation on animals.

The second mode is for the muscular or visceral nerves which is possible to isolate by endoscopic dissection. The electrode 16, 26, 36, 66, 80, 110 or 130 can be made in Nickel Titanium (Nitinol) which is a memory alloy. This material has the property to conserve the shape memory depending of the temperature. At a low temperature in the order of 4 to 5 degrees C., it becomes soft and deformable and at the body temperature it returns to its initial shape. That is of great advantage to put in place the electrode 16, 26, 36, 66, 80, 110 or 130 and in addition this material is super elastic which guarantee an excellent contact with the nerve.

From the foregoing description it will be apparent, that the electrodes 16, 26, 36, 66, 80, 110 or 130 of the present invention and the endoscopic method of implantation of the monopolar electrode 16, 26, 36, 66, 80, 110 or 130 has a number of advantages, some of which have been described above and others of which are inherent in the method and monopolar electrodes of the present invention.

Also, it will be understood that modifications can be made to the method and monopolar electrodes of the present invention without departing from the teachings of the present invention. Accordingly, the scope of the present invention is only to be limited as necessitated by the accompanying claims.

What is claimed is:

1. A monopolar electrode assembly for use in selective electro stimulation of a fascicle in a nerve bundle comprising an uncoated end portion of a wire conductor which is positioned adjacent an insulated base support structure adapted to be positioned adjacent a fascicle in a nerve bundle.

2. The monopolar electrode assembly of claim 1 wherein said base support structure has the general shape of a conduit strap or clamp.

3. The monopolar electrode assembly of claim 1 wherein said base support structure has the general shape of curved generally flat strip.

4. The monopolar electrode assembly of claim 1 wherein said base support structure is made of insulated nickel titanium alloy which is soft below the temperature of a human body and which forms a curled spiral shape at the temperature of a human body.

5. The monopolar electrode assembly of claim 1 wherein said base support structure is a clamp in the shape of a pipe hanger having a generally cylindrical portion and two radially extending leg portions.

6. The monopolar electrode assembly of claim 5 wherein said extending leg portions have openings for receiving sutures for securing said clamp to adjacent tissue.

7. The monopolar electrode assembly of claim 5 wherein said pipe hanger shaped base support structure includes an inner layer made of nickel titanium alloy and an outer insulating layer.

8. The monopolar electrode assembly of claim 6 wherein an inner electrode layer is positioned within the base support structure and has a generally flat arcuate shape for contacting a fascicle.

9. The monopolar electrode assembly of claim 7 wherein said inner electrode layer is made of platinum.

10. The monopolar electrode assembly of claim 7 wherein said inner electrode is made of a nickel titanium alloy.

11. The monopolar electrode assembly of claim 1 wherein said base support structure is generally cylindrical in shape and includes an outer generally cylindrical insulating layer and an inner metal layer made of a nickel titanium alloy.

12. The monopolar electrode assembly of claim 11 wherein an inner electrode layer is positioned within said base support structure.

13. The monopolar electrode assembly of claim 12 wherein said inner electrode layer is made of platinum.

14. The monopolar electrode assembly of claim 11 wherein said base support structure and said inner electrode layer comprise an electrode assembly.

15. The monopolar electrode assembly of claim 12 wherein said electrode assembly is open in the form of a C-shape under the temperature of a human body so that said electrode assembly can be positioned about a fascicle and will close about the fascicle when heated to the temperature of a human body.

16. The monopolar electrode assembly of claim 1 wherein said elrctrode assembly is an open cuff electrode assembly in the shape of a pipe clamp including said base support structure which is made of an insulating material and comprises a partially cylindrical portion with a longitudinal slot therein and opposed wing portions which extend laterally outwardly from the slot and from the cylindrical portion, said electrode is a flat curved platinum electrode which is mounted on an inner cylindrical surface of the partially cylindrical portion in positioned to make electrical contact with a nerve inserted into the partially cylindrical portion and a wire conductor extends through the partially cylindrical portion near one wing portion and is electrically connected to the flat platinum electrode.

17. The monopolar electrode assembly of claim 16 wherein a removable rod or skirt is fixed to each wing for being gripped to pull the wings away from the slot to expand the slot to enable a nerve to be inserted through the expanded slot 86 into the patially cylindrical portion and such that when the rods or skirts are released the cylindrical portion gripps the nerve inserted therein and presses the platinum electrode against the nerve.

18. The monopolar electrode assembly of claim 1 wherein said electrode assembly is a semi-cuff monopolar electrode assembly which includes an insulating base support structure that is made of an insulating material and includes a partially cylindrical portion with a longitudinal slot thereinand one wing extends from a side of the partially cylindrical portion near the slot, and said monopolar electrode is a flat curved platinum electrode which is mounted on an inner surface 6 of the partially cylindrical portion and a wire conductor 8 extends through the partially cylindrical portion adjacent the wing for electrical connection to the flat platinum electrode.

19. The monopolar electrode of claim 18 wherein a rod or skirt is removably mointed to the wing and a second rod or skirt s removably mountedd to the cylindrical portion near the slot on a side of the partially cylindrical portion which is on the other side of the slot 6 from where the wing is fixed to the partially cylindrical portion, the rods or skirts being gripped and pulled to open or expand the slot to enable a nerve to be inserted through the expanded slot into the partially cylindrical portion and when released the partially cylindrical portion is allowed to grip the nerve and urge the curved flat platinum electrode against the nerve.

20. The monopolar electrode assembly of claim 1 wherein said electrode assembly is a lasso monopolar electrode assembly including a flat but bendable platinum electrode electrically connected to a coiled wire conductor by a thin wire extending from the flat platinum electrode into the coiled wire conductor, the base support structure comprising an insulating silicon flap, said silicon flap has a narrowed end portion which is adapted to be pulled through a silicon collar which extends around a narrowed portion of the silicon flap, the platinum wire being on the inside of the silicon flap and the narrowed end portion is adapted to be pulled through the collar to lasso a nerve that has been inserted in a loop created by the flat electrode and the silicon flap and press the flat curled flat electrode against the nerve.

21. The monopolar electrode assembly of claim 20 wherein narrowed end portion has serrations on opposite side edges thereof and opposite sides of the collar have slits therein for being engaged by the serrations to lock the narrowed end portion in the collar

22. The monopolar electrode assembly of claim 20 wherein said flat electrode is approximately 50 microns thick and said thin wire conductor is approximately 0.005 mm in diameter.

23. A method for locating a nerve fascicle to be stimulated and inserting an electrode adjacent the nerve fascicle for stimulating same comprising the steps of: making an incision in a body adjacent the location of a desired nerve bundle; inserting an endoscope equipped with optical zoom into the body through the incision; dissecting gently the epineurium surrounding a nerve and electrically stimulating same with an electrode and noting the muscle that responds to the stimulation; repeating; the last step with other nerves until a target nerve and associated muscle is located; and inserting a monpolar electrode adjacent the target nerve for subsequent connection to a neurposthesis system.

24. The method of claim 23 wherein the insertion is made through a stomach wall and the method includes the step of first inflating the stomach with air.

25. The method of claim 23 wherein the target nerve is one of the appropriate nerves for the lower limb muscles, for the visceral organs including the bladder, colon and rectum or for the penis.
